(19) United States
(12) Patent Application Publication
(10) Pub. No.: US 2023/0210882 A1
(43) Pub. Date: Jul. 6, 2023

(54) CONJUGATE GROUP AND CONJUGATE

(71) Applicant: MEDSHINE DISCOVERY INC., Nanjing, Jiangsu (CN)

(72) Inventors: Ke AN, Shanghai (CN); Fei SUN, Shanghai (CN); Charles Z. DING, Shanghai (CN); Shuhui CHEN, Shanghai (CN)

(73) Assignee: MEDSHINE DISCOVERY INC., Nanjing, Jiangsu (CN)

(21) Appl. No.: 18/001,429

(22) PCT Filed: Jun. 10, 2021
(86) PCT No.: PCT/CN2021/099405

§ 371 Date: Dec. 9, 2022

(30) Foreign Application Priority Data
Jun. 10, 2020 (CN) 202010522407.6
Dec. 21, 2020 (CN) 202011524307.3

(51) Int. Cl. A61K 31/7088; A61K 47/54; C12N 15/113

(52) U.S. Cl. CPC A61K 31/7088; A61K 47/54; C12N 15/113

(57) ABSTRACT

The present application relates to a novel conjugate group linkable to a compound (such as a therapeutic compound), for use in directing the compound to a target in the body. The conjugate group disclosed herein can enable an expression-inhibitory oligonucleotide (such as a RNAi reagent) to target liver cells to regulate gene expression. The conjugate group disclosed herein has a variety of applications when linked to expression-inhibitory oligonucleotides, comprising applications in therapy, diagnosis, target verification, and genome development. A composition comprising the conjugate group disclosed herein can mediate expression of target nucleic acid sequences in liver cells (such as hepatocytes) when linked to expression-inhibitory oligonucleotides, and can be used for treatment of diseases or disorders responding to the gene expression or activity of cells, tissues, or organisms.

This application claims the priority of: CN202010522407.6, filed on Jun. 10, 2020; CN202011524307.3, filed on Dec. 21, 2020.

### FIELD OF THE INVENTION

The present disclosure relates to a novel conjugate group and use thereof. The conjugate group disclosed herein can be linked to a compound (e.g., a therapeutic agent) to direct the compound to a target in vivo.

### BACKGROUND OF THE INVENTION

Many compounds need to be delivered to a specific location (e.g., to desired cell(s)) to have a therapeutic effect or to be useful for diagnostic purposes, particularly when attempting to deliver a therapeutic compound in vivo. Further, the ability to efficiently deliver a compound to a specific location can limit or potentially eliminate unintended consequences (e.g., off-target effects) that may be caused by administration of the compound. One method to facilitate delivery of a compound, such as a therapeutic agent, to a desired location in vivo, is by linking or attaching the compound to a conjugate group.

One class of therapeutic agents that can be targeted using conjugate groups is oligonucleotides. Oligonucleotides comprising nucleotide sequences at least partially complementary to a target nucleic acid have been shown to alter the function and activity of the target both in vitro and in vivo. When delivered to a cell containing a target nucleic acid (e.g., mRNA), oligonucleotides have been shown to modulate the expression of the target, resulting in altered transcription or translation of the target nucleic acid. In certain instances, oligonucleotides can reduce the expression of gene by inhibiting a nucleic acid target and/or triggering the degradation of a target nucleic acid.

If the target nucleic acid is mRNA, one mechanism by which an expression-inhibitory oligonucleotide can modulate the expression of the mRNA target is through RNA interference. RNA interference is a biological process by which RNA or RNA-like molecules (e.g., chemically modified RNA molecules) are able to silence gene expression through degradation. The process of post-transcriptional gene silencing is thought to be an evolutionarily-conserved cellular defense mechanism used to prevent the expression of foreign genes.

Synthetic RNA and RNA-like molecules have been shown to elicit RNA interference in vivo. For example, Elbashir et al. (Nature 2000, 411, 494-98) describes RNAi induced by introduction of duplexes of synthetic 21-nucleotide RNA molecules in cultured mammalian cells. The types of synthetic RNA or RNA-like molecules that can trigger the RNAi response mechanism may comprise modified nucleotides and/or one or more non-phosphodiester linkages.

Meier et al. (J. Mol. Biol. 2000, 300, 857-65) reported an acetylgalactosamine (GalNAc) group that can bind tightly to the anti-asialoglycoprotein receptor (ASGPR) highly expressed in liver cells, and the co-crystal structure upon binding. Khorev et al. (Bioorg. Med. Chem. 2008. 16, 5216-31) reported the use of this group for targeted delivery of fluorescent chromophores to hepatocytes. Prakash et al. (J. Med. Chem. 2016, 59, 2718-33) and WO2009/073809 reported the use of GalNAc delivery platform to deliver antisense nucleotides and siRNAs to the liver and the achievement of corresponding gene silencing, respectively. Thus, GalNAc, a structural unit, has promising applications in the delivery of macromolecules to liver cells.

### SUMMARY OF THE INVENTION

The present disclosure relates to a novel conjugate group linkable to a compound (e.g., a therapeutic agent), for use in directing the compound to a target in vivo. The conjugate group disclosed herein can enable an expression-inhibitory oligonucleotide (e.g., an RNAi reagent) to target liver cells so as to regulate gene expression.

The conjugate group disclosed herein has a variety of applications when linked to expression-inhibitory oligonucleotides, comprising applications in therapy, diagnosis, target verification, and genome development. A composition comprising the conjugate group disclosed herein can mediate the expression of target nucleic acid sequences in liver cells (e.g., hepatocytes) when linked to expression-inhibitory oligonucleotides. This can be used for treatment of diseases or disorders responding to the gene expression or activity of cells, tissues, or organisms.

Thus, in the first aspect, the present disclosure provides a conjugate group, which has a structure of formula (I),

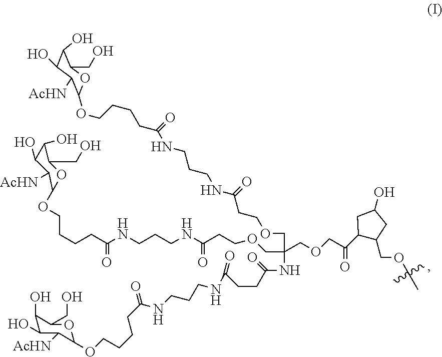

wherein n is selected from an integer of 8 to 12.

In some embodiments of the first aspect, the conjugate group may have the following structure:

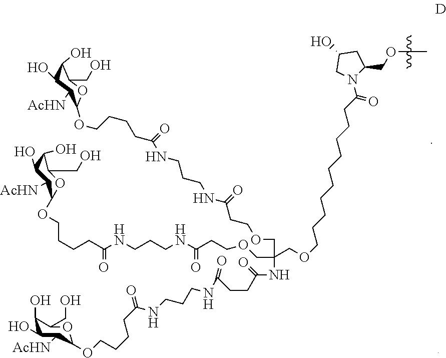

In the second aspect, the present disclosure provides a conjugate comprising the conjugate group according to the first aspect of the present disclosure, and a therapeutic agent linked to the conjugate group.

In some embodiments of the second aspect, the therapeutic agent in the above conjugate is an expression-inhibitory oligonucleotide.

In some embodiments of the second aspect, the expression-inhibitory oligonucleotide in the above conjugate is an RNAi reagent.

In some embodiments of the second aspect, the RNAi reagent in the above conjugate contains one or more modified nucleotides.

In some embodiments of the second aspect, the RNAi reagent in the above conjugate is a double-stranded siRNA containing a sense strand and an antisense strand.

In some embodiments of the second aspect, the double-stranded siRNA in the above conjugate is linked to the conjugate group at the 5′ end of its sense strand.

In some embodiments of the second aspect, the expression-inhibitory oligonucleotide in the above conjugate is linked to the conjugate group via a phosphate, phosphorothioate, or phosphonate group.

In some embodiments of the second aspect, the phosphorothioate moiety of the above conjugate or expression-inhibitory oligonucleotide includes (R)- and (S)-enantiomers, diastereomers, and/or racemic mixtures thereof.

In some embodiments of the second aspect, the above disclosure also provides salts of the conjugate.

In some embodiments of the second aspect, the salts as described above are selected from base addition salts, acid addition salts and combinations thereof.

In some embodiments of the second aspect, the above base addition salts are selected from sodium, potassium, calcium, ammonium, organic amines, magnesium salts and combinations thereof, and the acid addition salts are selected from salts of inorganic acids, salts of organic acids and combinations thereof.

In some embodiments of the second aspect, the above inorganic acids are selected from hydrochloric acid, hydrobromic acid, nitric acid, carbonic acid, bicarbonate, phosphoric acid, monohydrogen phosphate, dihydrogen phosphate, sulfuric acid, bisulfate, hydroiodic acid, phosphorous acid and combinations thereof, and the organic acids are selected from acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, methanesulfonic acid and combinations thereof.

In the third aspect, the present disclosure provides a compound having a structure of formula (II) or formula (III).

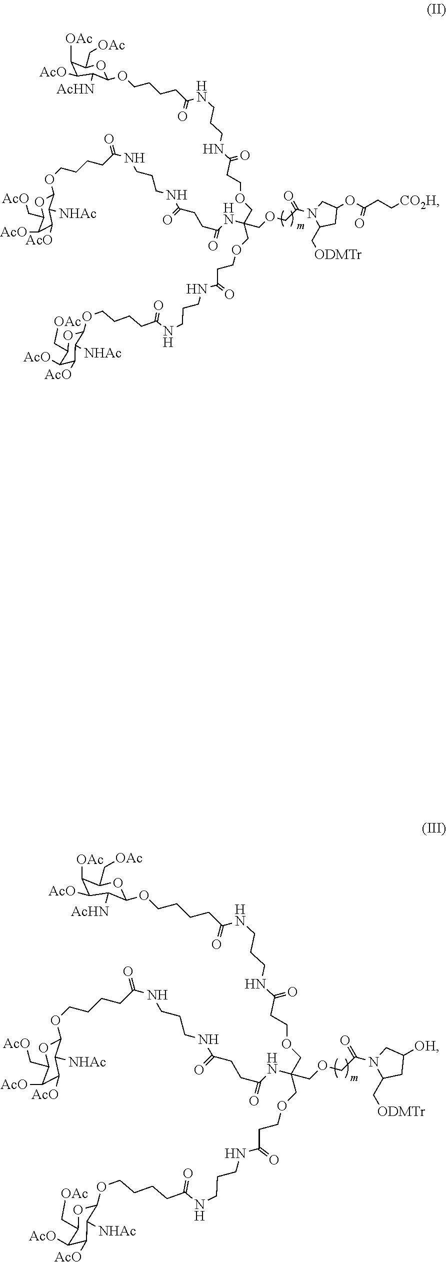

wherein m is selected from an integer of 8 to 12.

In some embodiments of the third aspect, the compound may have the following structure

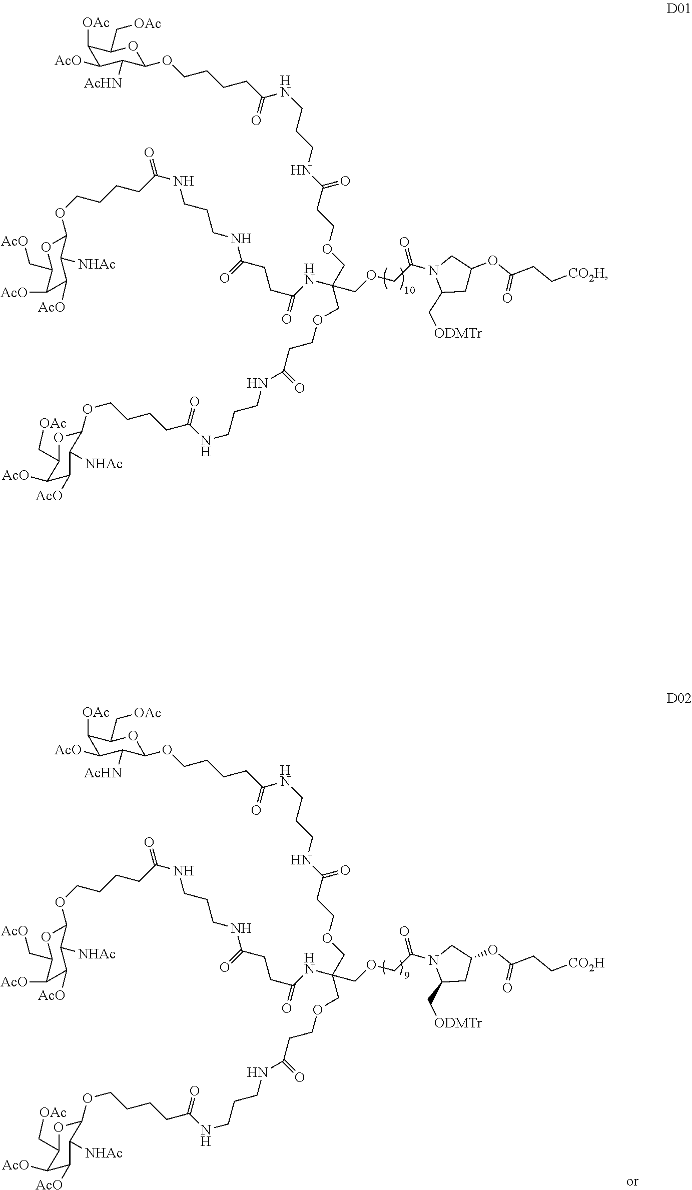

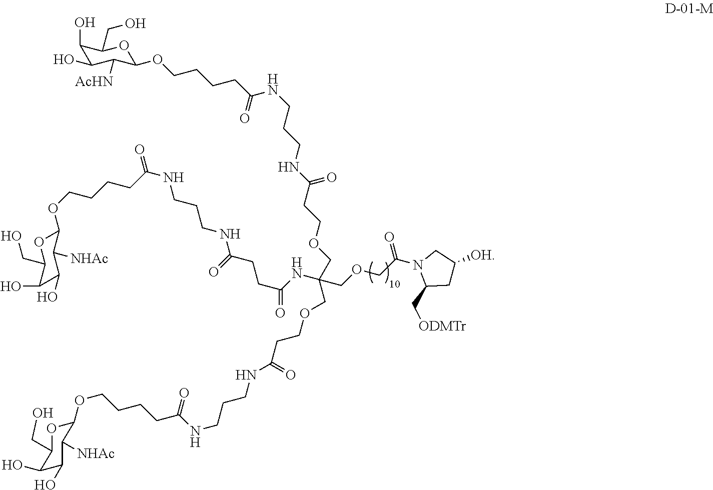

In the fourth respect, the present disclosure provides a pharmaceutical composition comprising the conjugate according to the second aspect of the present disclosure, and a pharmaceutically acceptable carrier or excipient.

In the fifth aspect, the present disclosure provides a method for inhibiting the expression of a target nucleic acid in a subject in need thereof, which comprises a step of administering to the subject the conjugate according to the second aspect of the present disclosure or the pharmaceutical composition according to the fourth aspect of the present disclosure.

In some embodiments of the fifth aspect, the target nucleic acid in the method is a nucleic acid from a virus. The virus may be, for example, a virus that causes a liver disease, such as a hepatitis B virus.

In the sixth aspect, the present disclosure provides a method of treating a disease, which comprises a step of administering to the subject the conjugate according to the second aspect of the present disclosure or the pharmaceutical composition according to the fourth aspect of the present disclosure.

In the seventh aspect, the present disclosure provides a use of the conjugate according to the second aspect of the present disclosure or the pharmaceutical composition according to the fourth aspect of the present disclosure in the manufacture of a medicament for the treatment of diseases.

In the eighth aspect, the present disclosure provides the conjugate according to the second aspect of the present disclosure or the pharmaceutical composition according to the fourth aspect of the present disclosure, for use in treating diseases.

In some embodiments of the seventh and eighth aspects, the disease is viral infection.

In some embodiments of the seventh and eighth aspects, the disease is liver disease.

In some embodiments of the seventh and eighth aspects, the disease is hepatitis B.

### Definition and Description

Unless otherwise specified, the following terms and phrases used herein are intended to have the following meanings. A specific term or phrase should not be considered indefinite or unclear in the absence of a particular definition, but should be understood in the sense understood by one of ordinary skill in the art. When a trade name appears herein, it is intended to refer to its corresponding commodity or active ingredient thereof.

The conjugate group described in the present disclosure can enhance delivery of a therapeutic agent to a specific target location (e.g., a specific organ or tissue) in a subject such as a human or animal. In some embodiments of the present disclosure, the conjugate group can enhance targeted delivery of an expression-inhibitory oligonucleotide. In some embodiments of the present disclosure, the conjugate group can enhance delivery of an expression-inhibitory oligonucleotide to the liver.

The conjugate groups described in the present disclosure may be directly or indirectly linked to a compound, such as a therapeutic agent, for example, an expression-inhibitory oligonucleotide, e.g., the 3′ or 5′ end of the expression-inhibitory oligonucleotide. In some embodiments of the present disclosure, the expression-inhibitory oligonucleotide comprises one or more modified nucleotides. In some embodiments of the present disclosure, the expression-inhibitory oligonucleotide is an RNAi reagent, such as a double-stranded RNAi reagent comprising a sense strand and an antisense strand. In some embodiments of the present disclosure, the conjugate group disclosed herein is linked to the 5′ end of the sense strand of a double-stranded RNAi reagent. In some embodiments, the conjugate group disclosed herein is linked to the expression-inhibitory oligonucleotide reagent via a phosphate, phosphorothioate, or phosphonate group at the 5′ end of the sense strand of a double-stranded RNAi reagent.

As used herein, the term “linked” when referring to the connection between two molecules means that two molecules are joined by a covalent bond or that two molecules are associated via a non-covalent bond (e.g., a hydrogen bond or an ionic bond).

As used herein, an “oligonucleotide” is a nucleotide sequence containing 10-50 nucleotides or nucleotide base pairs. In some embodiments of the present disclosure, an oligonucleotide has a nucleobase sequence that is at least partially complementary to a coding sequence in a target nucleic acid or a target gene expressed in a cell. The nucleotides may optionally be modified. In some embodiments of the present disclosure, the oligonucleotide, upon delivery to a cell expressing a gene, is able to inhibit the expression of the underlying gene, and is referred to herein as “expression-inhibitory oligonucleotide”, which can inhibit gene expression in vitro or in vivo. “Oligonucleotides” include, but are not limited to: single-stranded oligonucleotides, single-stranded antisense oligonucleotides, short interfering RNAs (siRNAs), double-stranded RNAs (dsRNAs), micro RNAs (miRNAs), short hairpin RNAs (shRNAs), ribozymes, interfering RNA molecules, and Dicer substrates.

As described herein, an “RNAi reagent” means a reagent that contains an RNA or RNA-like (e.g., chemically modified RNA) oligonucleotide molecule that is capable of degrading or inhibiting translation of messenger RNA (mRNA) transcripts of a target mRNA in a sequence-specific manner. As described herein, RNAi reagents may operate by the RNA interference mechanism (i.e., inducing RNA interference through interaction with the RNA interference pathway machinery (RNA-induced silencing complex or RISC) of mammalian cells), or may function by any alternative mechanism(s) or pathway(s). While the RNAi reagents described herein operate primarily by the RNA interference mechanism, the disclosed RNAi reagents are not bound by or limited to any particular pathway or mechanism of action. RNAi reagents include, but are not limited to: single-stranded oligonucleotides, single-stranded antisense oligonucleotides, short interfering RNAs (siRNAs), double-stranded RNAs (dsRNAs), micro RNAs (miRNAs), short hairpin RNAs (shRNAs), and Dicer substrates. The RNAi reagent described herein comprises an oligonucleotide having a strand that is at least partially complementary to a targeted mRNA. In some embodiments of the present disclosure, the RNAi reagent described herein is double-stranded, and comprises an antisense strand and a sense strand that is at least partially complementary to the antisense strand. RNAi reagents may comprise modified nucleotides and/or one or more non-phosphodiester linkages. In some embodiments of the present disclosure, the RNAi reagent described herein is single-stranded.

As described herein, the term “single-stranded oligonucleotide” means a single-stranded oligonucleotide having a sequence at least partially complementary to a target mRNA, which is capable of hybridizing with the target mRNA through hydrogen bonding under mammalian physiological conditions (or comparable conditions in vitro). In some embodiments of the present disclosure, a single-stranded oligonucleotide is a single-stranded antisense oligonucleotide.

The short interfering RNA (siRNA) of the present disclosure is a class of RNA molecules with a length of 20-25 base pairs, which are similar to miRNA and operate in an RNA interference (RNAi) pathway. The short interfering RNA (siRNA) of the present disclosure interferes with the translation of mRNA of specific genes complementary to nucleotide sequences, resulting in degradation of mRNA. The short interfering RNA (siRNA) described in the present disclosure includes double-stranded siRNA (containing a sense strand and an antisense strand) and single-stranded siRNA (an antisense strand only).

As described herein, the term “silence”, “reduce”, “inhibit”, “down-regulate”, or “knockdown”, when referring to expression of a given gene, means that the expression of the gene, as measured by the level of RNA transcribed from the gene or the level of polypeptide, protein or protein subunit translated from the mRNA in a cell, group of cells, tissue, or subject in which the gene is transcribed, is reduced when the cell, group of cells, tissue, or subject is treated with oligonucleotides linked to the conjugate groups described herein as compared to a second cell, group of cells, tissue, or subject that has not or have not been so treated.

As described herein, the term “sequence” or “nucleotide sequence” means a succession or sequence of nucleobases or nucleotides described with a sequence of letters using the standard nucleotide nomenclature.

The HBV gene described in the present disclosure refers to a gene with a DNA sequence indicated by Genbank registration number NC_003977.1. The gene indicated by Genbank registration number NC_003977.1 is the complete genome of HBV.

In some embodiments, the double-stranded siRNA analogue may target the X opening reading frame (X ORF) of HBV.

In a further embodiment, the double-stranded siRNA analogue may target the S ORF of HBV.

In a further embodiment, the double-stranded siRNA analogue may target the P ORF of HBV.

The “modification” of nucleotides described in the present disclosure includes, but is not limited to, methoxy modification, fluorinated modification, attachment of phosphorothioate group, etc. The sequence described in the present disclosure may include the “further modified sequence” listed in Table 1 below.

In the present disclosure, unless otherwise specified, the uppercase letters C, G, U, and A indicate the base composition of the nucleotide; the lowercase letters c, g, u, and a respectively indicate that the nucleotide represented by the corresponding uppercase letter is modified by a methoxy group; underline indicates that the nucleotide represented by an uppercase letter is modified by fluorination; and the separation dot “⋅” indicates a phosphorothioate linkage between two nucleotide residues adjacent to the left and right of the separation dot “⋅”. For example, “a⋅g” indicates that a and g residues are linked by a phosphorothioate group.

The nucleotide modified by fluorination described in the present disclosure refers to a nucleotide formed by replacement of the hydroxyl group at the ribosyl 2′ position of the nucleotide by fluorine, and the nucleotide modified by a methoxy group refers to a nucleotide formed by replacement of the 2′-hydroxyl group of the ribosyl group by a methoxy group.

In the present disclosure, “complementary” has the meaning known to those of skill in the art, i.e., in double-stranded nucleic acid molecules, the bases of one strand are paired with the bases on the other strand in a complementary manner. The purine base adenine (A) is always paired with the pyrimidine base uracil (U); and the purine base guanine (C) is always paired with the pyrimidine base cytosine (G). Each base pair includes one purine and one pyrimidine. Where an adenine on one strand is always paired with a uracil on the other strand and a guanine is always paired with a cytosine, the two strands are said to be complementary to each other and the sequence of one strand can be deduced from the sequence of its complementary strand.

Compounds disclosed herein may be present in a specific geometric or stereoisomeric form. The present disclosure contemplates all such compounds, including (R)- and (S)-enantiomers, diastereoisomer, and a racemic mixture and other mixtures, for example, a mixture enriched in enantiomer or diastereoisomer, all of which are encompassed within the scope disclosed herein. The substituent such as alkyl may have an additional asymmetric carbon atom. All these isomers and mixtures thereof are encompassed within the scope disclosed herein.

Unless otherwise specified, the term “enantiomer” or “optical isomer” refers to stereoisomers that are in a mirrored relationship with each other.

Unless otherwise specified, the term “diastereomer” refers to a stereoisomer in which two or more chiral centers of are contained in a molecule and is in a non-mirrored relationship between molecules.

Unless otherwise specified, a wedged solid bond ( [symbol] ) and a wedged dashed bond ( [symbol] ) indicate the absolute configuration of a stereocenter; a straight solid bond ( [symbol] ) and a straight dashed bond ( [symbol] ) indicate the relative configuration of a stereocenter; a wavy line ( [symbol] ) indicates a wedged solid bond ( [symbol] ) or a wedged dashed bond ( [symbol] ) or a wavy line ( [symbol] ) indicates a straight solid bond ( [symbol] ) and/or a straight dashed bond ( [symbol] ).

Unless otherwise specified, the term “enriched in one isomer”, “isomer enriched”, “enriched in one enantiomer” or “enantiomeric enriched” means that the content of one isomer or enantiomer is less than 100%, and the content of the isomer or enantiomer is 60% or more, or 70% or more, or 80% or more, or 90% or more, or 95% or more, or 96% or more, or 97% or more, or 98% or more, or 99% or more, or 99.5% or more, or 99.6% or more, or 99.7% or more, or 99.8% or more, or 99.9% or more.

Unless otherwise specified, the term “isomer excess” or “enantiomeric excess” means the difference between the relative percentages of two isomers or two enantiomers. For example, if one isomer or enantiomer is present in an amount of 90% and the other isomer or enantiomer is present in an amount of 10%, the isomer or enantiomeric excess (ee value) is 80%.

Optically active (R)- and (S)-isomer, or D and L isomer can be prepared using chiral synthesis or chiral reagents or other conventional techniques. If one kind of enantiomer of certain compound disclosed herein is to be obtained, the pure desired enantiomer can be obtained by asymmetric synthesis or derivative action of chiral auxiliary followed by separating the resulting diastereomeric mixture and cleaving the auxiliary group. Alternatively, when the molecule contains a basic functional group (e g, amino) or an acidic functional group (e.g., carboxyl), the compound reacts with an appropriate optically active acid or base to form a salt of the diastereomeric isomer which is then subjected to diastereomeric resolution through the conventional method in the art to afford the pure enantiomer. In addition, the enantiomer and the diastereoisomer are generally isolated through chromatography which uses a chiral stationary phase and optionally combines with a chemical derivative method (e.g., carbamate generated from amine) Compounds disclosed herein may contain an unnatural proportion of atomic isotopes at one or more of the atoms that make up the compounds. For example, a compound may be labeled with a radioisotope such as tritium (³H), iodine-125 (¹²⁵I) or C-14(¹⁴C). For another example, hydrogen can be replaced by heavy hydrogen to form a deuterated drug. The bond between deuterium and carbon is stronger than that between ordinary hydrogen and carbon. Compared with undeuterated drugs, deuterated drugs have advantages of reduced toxic side effects, increased drug stability, enhanced efficacy, and prolonged biological half-life of drugs. All changes in the isotopic composition of compounds disclosed herein, regardless of radioactivity, are included within the scope of the present disclosure.

The term “salt” means a salt of a compound disclosed herein that is prepared by reacting the compound having a specific substituent disclosed herein with a relatively non-toxic acid or base. When compounds disclosed herein contain a relatively acidic functional group, a base addition salt can be obtained by bringing the compound into contact with a sufficient amount of base in a pure solution or a suitable inert solvent. The pharmaceutically acceptable base addition salt includes a salt of sodium, potassium, calcium, ammonium, organic amine or magnesium or similar salts. When compounds disclosed herein contain a relatively basic functional group, an acid addition salt can be obtained by bringing the compound into contact with a sufficient amount of acid in a pure solution or a suitable inert solvent. Examples of the pharmaceutically acceptable acid addition salt include an inorganic acid salt, wherein the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, wherein the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benzenesulfonic acid, p-toluenesulfonic acid, citric acid, tartaric acid, and methanesulfonic acid, and the like; and an salt of amino acid (e.g., arginine and the like), and a salt of an organic acid such as glucuronic acid and the like. Certain specific compounds disclosed herein contain both basic and acidic functional groups and can be converted to any base or acid addition salt.

The salt disclosed herein can be prepared from the parent compound that contains an acidic or basic moiety by conventional chemical methods. Generally, such salt can be prepared by reacting the free acid or base form of the compound with a stoichiometric amount of an appropriate base or acid in water or an organic solvent or a mixture thereof.

The compounds of the present disclosure may be prepared by a variety of synthetic methods known to those skilled in the art, including the specific embodiments listed below, the embodiments formed by the specific embodiments listed below combined with other chemical synthesis methods, and equivalent substitutions known to those skilled in the art. Alternative embodiments include, but are not limited to, examples of the present disclosure.

The solvents used in the present disclosure are commercially available.

Unless otherwise specified, the solvent ratios used for column chromatography and thin-layer silica gel chromatography in this disclosure are all volume ratios.

List of abbreviations

| | |
|---|---|
| Ac | Acetyl |
| Boc | tert-Butoxycarbonyl |
| DMSO | Dimethylsulfoxide |
| DMT/DMTr | 4,4′-Dimethoxytriphenylmethyl |
| dsRNA | Double-stranded ribonucleic acid |
| EC₅₀ | Concentration for 50% of maximal effect |
| EDTA | Disodium Ethylenediaminetetraacetate |
| i-Pr | Isopropyl |
| Me | Methyl |
| Ms | Methanesulfonyl |
| Ph | Phenyl |
| p-HPLC | Preparative high performance liquid chromatography, |
| | used for purification of compounds |
| RNA | Ribonucleic acid |
| RNAi | Ribonucleic acid interference technique |
| siRNA | Short interfering ribonucleic acid |
| t-Bu | tert-butyl |
| Tris | Tris(hydroxymethyl)aminomethane |

Compounds are named according to general naming principles in the art or by ChemDraw® software, and commercially available compounds are named with their vendor directory names.

### DETAILED DESCRIPTION OF THE INVENTION

The following examples describe the present disclosure in detail, but they are not meant to limit the present disclosure in any way. Compounds disclosed herein can be prepared by a variety of synthetic methods well known to those skilled in the art, including the specific embodiments listed below, the embodiments formed by the specific embodiments listed below combined with other chemical synthesis methods, and equivalent substitutions known to those skilled in the art. Alternative embodiments include, but are not limited to the examples disclosed herein. It will be obvious to those skilled in the art to make various changes and improvements to the specific embodiments of this disclosure without departing from the spirit and scope of this disclosure.

### Example 1: Synthesis of D01

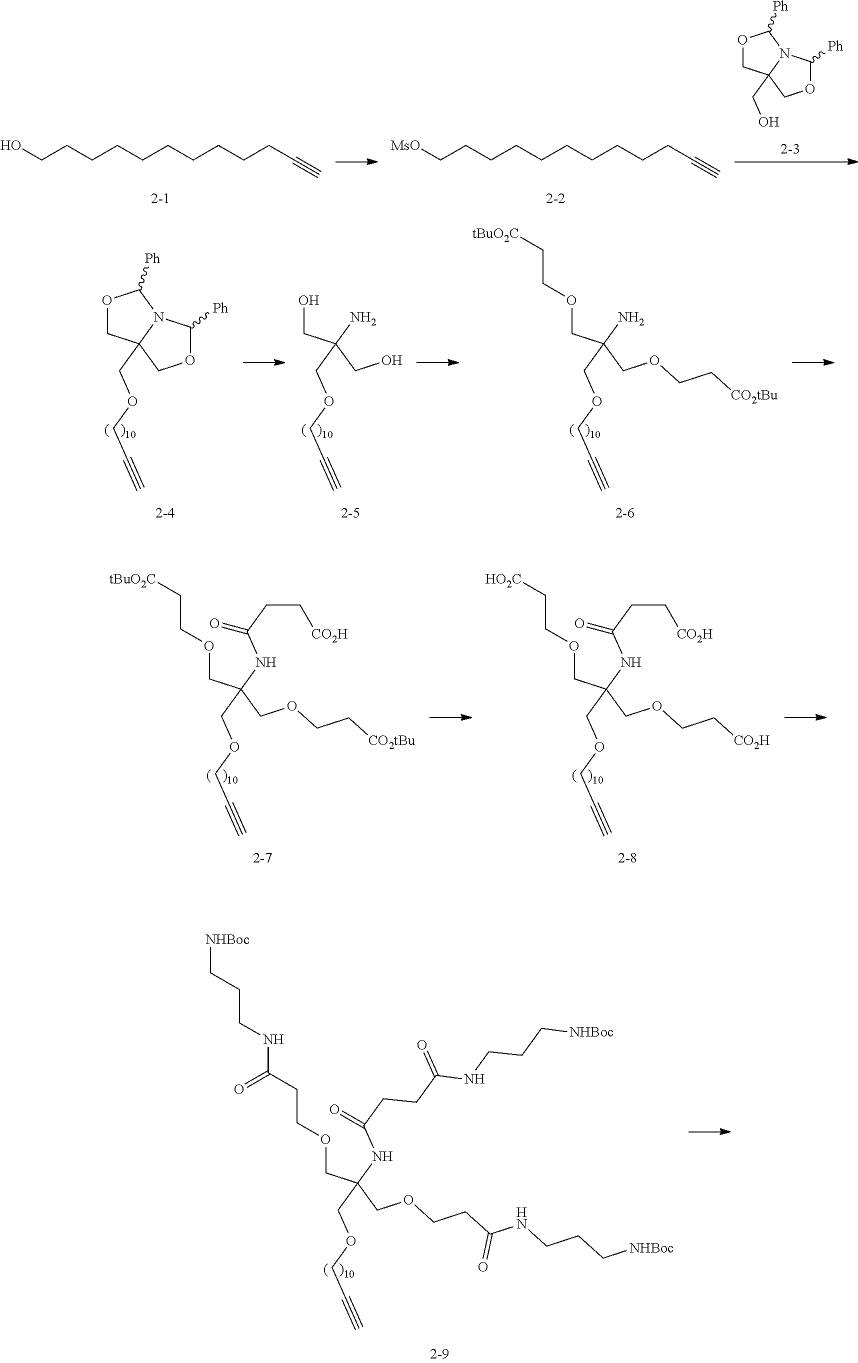

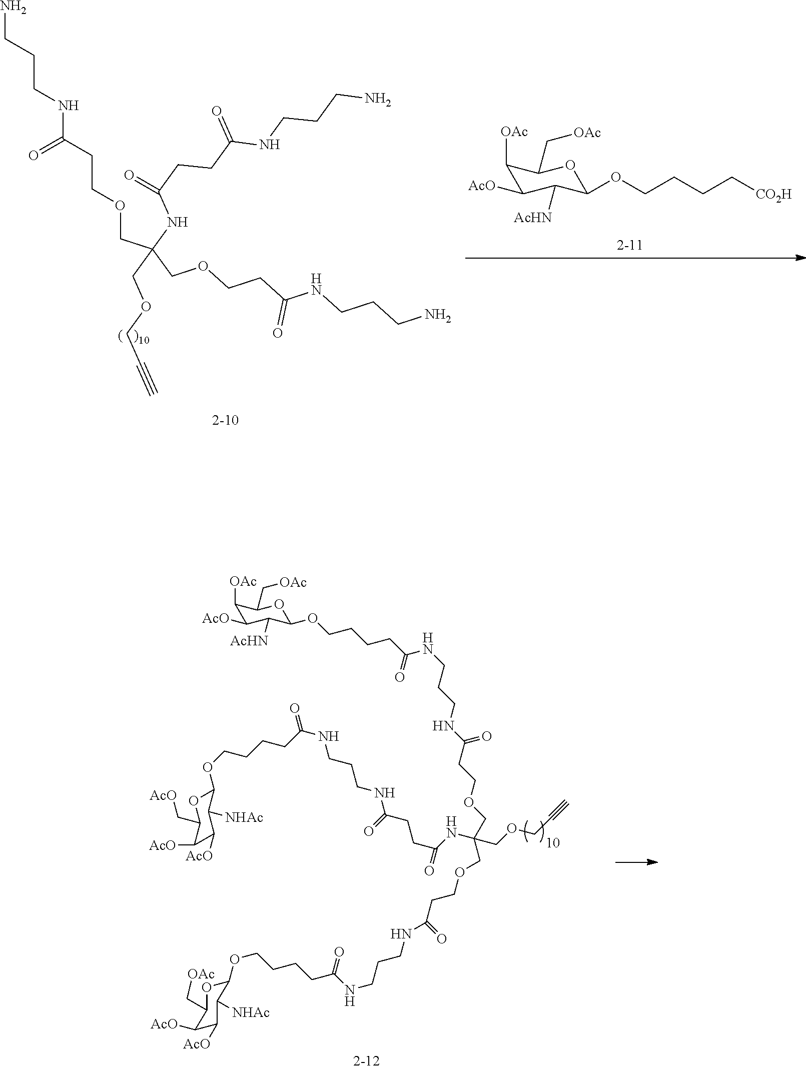

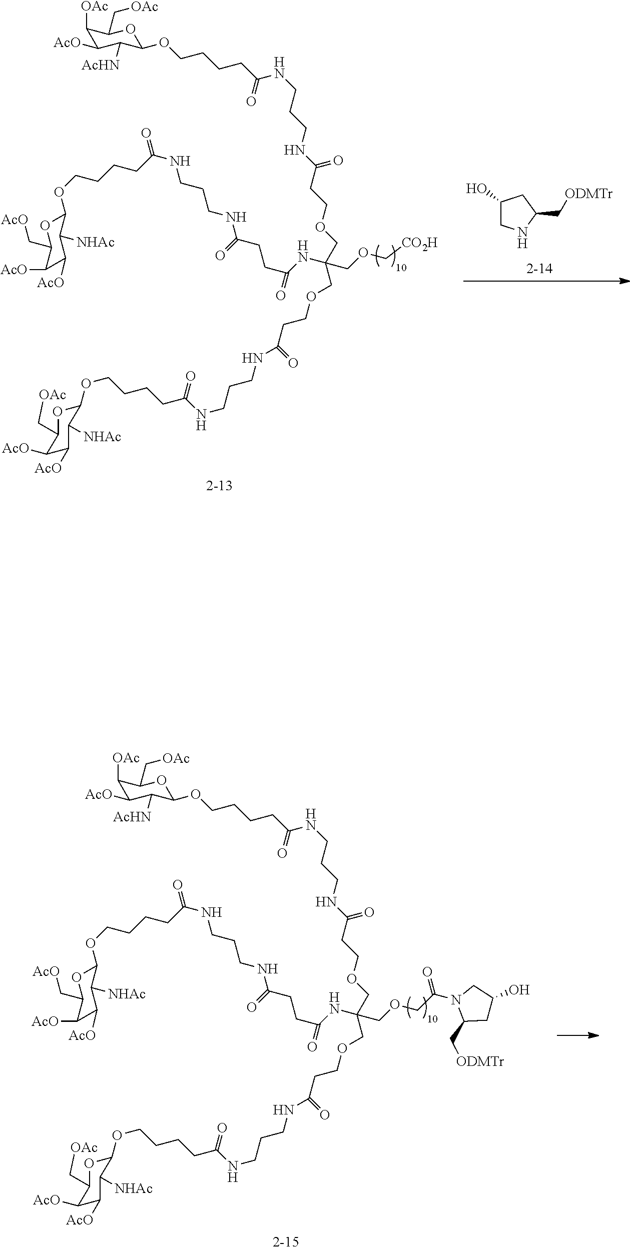

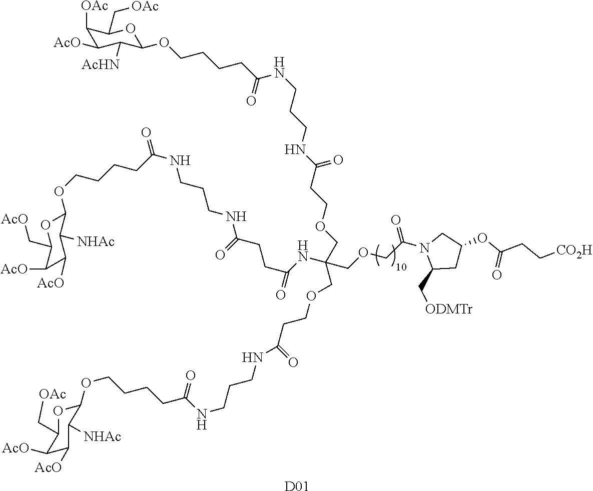

Step A: 11-Dodecyne-1-ol (25 g, 137.14 mmol) and triethylamine (16.65 g, 164.56 mmol) were dissolved in dichloromethane (250 mL), and methanesulfonyl chloride (18.85 g, 164.56 mmol) was added at 0° C. The mixture solution was stirred at 0° C. for 2 hours. The reaction solution was diluted with water (400 mL) and extracted with 800 mL (400 mL×2) of dichloromethane. The combined organic phase was washed with 400 mL (200 mL×2) of water and saturated brine, dried over anhydrous sodium sulfate, and filtered. The filtrate was concentrated under reduced pressure to give 2-2.

Step B: The compound of formula 2-3 (20 g, 67.26 mmol) was dissolved in N,N-dimethylformamide (200 mL), and sodium hydride (60% purity, 4.04 g, 100.89 mmol) was added at 0° C., followed by the compound of formula 2-2 (19.27 g, 73.99 mmol). The mixture was stirred at 25° C. for 16 h. The reaction solution was quenched with water (1 L) and extracted with 1.6 L (800 mL×2) of dichloromethane. The combined organic phase was washed with 800 mL (800 mL×1) of saturated brine, dried over anhydrous sodium sulfate, and filtered. The filtrate was concentrated under reduced pressure to give 2-4. ¹H NMR (400 MHz, DMSO-d₆): δ 7.63-6.89 (m, 10H), 5.64-5.52 (m, 2H), 4.27-4.01 (m, 2H), 3.98-3.77 (m, 2H), 3.72-3.18 (m, 4H), 2.23-2.14 (m, 2H), 1.98-1.92 (m, 1H), 1.54-1.23 (m, 16H).

Step C: The compound of formula 2-4 (48 g, 103.98 mmol) was dissolved in methanol (870 mL), and a solution of hydrogen chloride in methanol (4 mol/L, 400 mL, 1.6 mol) was added. The mixture solution was stirred at 30° C. for 2 h. To the reaction solution was added a solution of hydrogen chloride in methanol (4 mol/L, 350 mL, 1.4 mol). The mixture was stirred at 30° C. for 16 hours. The reaction solution was concentrated under reduced pressure, and 200 mL (100 mL×2) of trichloromethane was added. The mixture was concentrated under reduced pressure until a white solid appeared. Toluene (130 mL) and petroleum ether (130 mL) were added, and the mixture solution was stirred at 15° C. for 16 hours. The reaction solution was filtered through a Buchner funnel and the filter cake was collected and dried under vacuum to give a white solid. The white solid was dissolved in dichloromethane (50 mL), and aqueous solution (50 mL) of sodium hydroxide (6.59 g, 164.66 mmol) was added. The mixture was stirred at 20° C. for 1 hour. The reaction solution was diluted with water (500 mL) and extracted with 1 L (500 mL×2) of dichloromethane. The combined organic phase was dried over anhydrous sodium sulfate, and filtered. The filtrate was concentrated under reduced pressure to give 2-5.

Step D: To a mixture solution of the compound of formula 2-5 (23 g, 80.58 mmol) and sodium hydroxide (322.31 mg, 8.06 mmol) in dimethyl sulfoxide (70 mL) and water (6 mL) was added tert-butyl acrylate (22.72 g, 177.28 mmol), and the mixture was stirred at 25° C. under nitrogen for 16 h. The reaction solution was diluted with water (500 mL) and extracted with 1 L of ethyl acetate (500 mL×2). The combined organic phase was dried over anhydrous sodium sulfate, and filtered. The filtrate was concentrated under reduced pressure to give the crude product, which was purified by column chromatography (SiO₂, petroleum ether/ethyl acetate/ethanol (with 0.1% ammonia)=36/3/1 to 16/3/1) to give 2-6. ¹H NMR (400 MHz, DMSO-d₆): δ 3.60-3.54 (m, 4H), 3.32 (br s, 5H), 3.15 (s, 5H), 2.74-2.66 (m, 1H), 2.40 (t, J=6.0 Hz, 4H), 2.18-2.11 (m, 2H), 1.58-1.38 (m, 22H), 1.34-1.23 (m, 12H).

Step E: To a solution of the compound of formula 2-6 (24.5 g, 45.22 mmol) in dichloromethane (250 mL) were added triethylamine (9.15 g, 90.45 mmol) and succinic anhydride (6.79 g, 67.83 mmol), and the mixture was stirred at 20° C. for 16 hours. To the reaction solution were added dichloromethane (1 L) and hydrochloric acid (1 mol/L, 1 L), and the layers were separated. The organic phase was dried over anhydrous sodium sulfate and filtered. The filtrate was concentrated under reduced pressure to give 2-7. ¹H NMR (400 MHz, CDCl₃): δ 6.49-6.37 (m, 1H), 3.72 (s, 2H), 3.70-3.57 (m, 8H), 3.37 (t, J=6.7 Hz, 2H), 2.69-2.51 (m, 4H), 2.50-2.36 (m, 4H), 2.22-2.13 (m, 2H), 1.96-1.90 (m, 1H), 1.57-1.47 (m, 4H), 1.46-1.40 (m, 18H), 1.40-1.31 (m, 2H), 1.30-1.21 (m, 10H).

Step F: The compound of formula 2-7 (27.4 g, 42.69 mmol) was dissolved in formic acid (140 mL), and the mixture was stirred at 20° C. under nitrogen for 16 hours. The reaction solution was concentrated under reduced pressure. 300 mL (150 mL×2) of toluene was added. The mixture was concentrated under reduced pressure to give 2-8. ¹H NMR (400 MHz, CDCl₃): δ 9.79-9.22 (m, 3H), 6.44-6.23 (m, 1H), 3.88-3.43 (m, 10H), 3.39-3.20 (m, 2H), 2.77-2.31 (m, 8H), 2.15-2.06 (m, 2H), 1.87 (t, J=2.6 Hz, 1H), 1.48-1.28 (m, 6H), 1.26-1.12 (m, 10H).

Step G: The compound of formula 2-8 (22.6 g, 42.67 mmol), N,N-diisopropylethylamine (33.09 g, 256.03 mmol) and O-(7-azabenzotriazol-1-yl)-N,N,N,N-tetramethyluronium hexafluorophosphate (51.92 g, 136.55 mmol) were dissolved in N,N-dimethylformamide (250 mL), and tert-butyl N-(3-aminopropyl)carbamate (29.74 g, 170.69 mmol) was added. The mixture solution was stirred at 20° C. for 16 h. To the reaction solution were added dichloromethane (1 L) and hydrochloric acid (1 mol/L, 1 L), and the layers were separated. The organic phase was washed sequentially with 1 L of water (1 L x 1), 1 L of aqueous sodium bicarbonate (1 L x 1) and 1 L of saturated brine (1 L x 1), dried over anhydrous sodium sulfate, and filtered. The filtrate was concentrated under reduced pressure to give the crude product, which was purified by column chromatography (SiO₂, petroleum ether/ethyl acetate/ethanol=40/3/1 to 10/3/1) to give 2-9. ¹H NMR (400 MHz, CDCl₃): δ 7.22-6.79 (m, 3H), 6.77-6.44 (m, 1H), 5.45-5.00 (m, 3H), 3.86-3.73 (m, 2H), 3.72-3.63 (m, 4H), 3.62-3.45 (m, 4H), 3.41-3.32 (m, 2H), 3.32-3.20 (m, 6H), 3.19-3.03 (m, 6H), 2.56-2.47 (m, 4H), 2.47-2.39 (m, 4H), 2.21-2.12 (m, 2H), 1.95-1.90 (m, 1H), 1.70-1.57 (m, 6H), 1.56-1.47 (m, 4H), 1.46-1.38 (m, 29H), 1.30-1.25 (m, 10H).

Step H: The compound of formula 2-9 (15 g, 15.03 mmol) was dissolved in dichloromethane (114 mL), and trifluoroacetic acid (38 mL) was added. The mixture was stirred at 20° C. for 16 hours. The reaction solution was concentrated under reduced pressure, and 600 mL (250 mL×3) of a mixed solution of toluene and acetonitrile (toluene/acetonitrile=3/1) was added. The solution was concentrated under reduced pressure to give 2-10.

Step I: The compound of formula 2-11 (22.15 g, 49.50 mmol), N,N-diisopropylethylamine (7.75 g, 60.00 mmol), 1-hydroxy-7-azabenzotriazole (6.12 g, 45.00 mmol) and O-(7-azabenzotriazol-1-yl)-N,N,N,N-tetramethyluronium hexafluorophosphate (20.53 g, 54.00 mmol) were dissolved in N,N-dimethylformamide (90 mL). To this mixture solution was added a solution of the compound of formula 2-10 (tris(trifluoroacetate), 15.6 g, 15.00 mmol) and N,N-diisopropylethylamine (21.32 g, 165.00 mmol) in N,N-dimethylformamide (120 mL). The mixture solution was stirred at 20° C. for 16 hours. To the reaction solution were added dichloromethane (1.2 L) and hydrochloric acid (1 mol/L, 1 L), and the layers were separated. The organic phase was washed sequentially with 1 L of water (1 L x 1), 1 L of aqueous sodium bicarbonate (1 L x 1) and 1 L of saturated brine (1 L x 1), dried over anhydrous sodium sulfate, and filtered. The filtrate was concentrated under reduced pressure to give the crude product, which was purified by column chromatography (SiO₂, dichloromethane/methanol=100/1 to 10/1 to dichloromethane/ethanol=1/1) to give 2-12. ¹H NMR (400 MHz, DMSO-d6): δ 7.87-7.66 (m, 9H), 7.09 (s, 1H), 5.21 (d, J=3.4 Hz, 3H), 4.96 (dd, J=3.4, 11.3 Hz, 3H), 4.48 (d, J=8.5 Hz, 3H), 4.06-3.98 (m, 9H), 3.91-3.82 (m, 3H), 3.74-3.66 (m, 3H), 3.58-3.46 (m, 12H), 3.31 (br s, 3H), 3.07-2.98 (m, 12H), 2.71 (t, J=2.6 Hz, 1H), 2.33-2.22 (m, 8H), 2.16-2.12 (m, 2H), 2.10 (s, 9H), 2.04 (br t, J=7.1 Hz, 6H), 1.99 (s, 9H), 1.89 (s, 9H), 1.81-1.74 (m, 9H), 1.54-1.39 (m, 22H), 1.32 (br dd, J=4.5, 6.7 Hz, 2H), 1.24 (s, 10H).

Step J: The compound of formula 2-12 (1.00 g, 0.50 mmol) and N-methyl-N,N,N,N-tri-n-octylammonium chloride (20.35 mg, 50.35 μmol) were dissolved in a mixture of acetic acid (2.7 mL) and n-pentane (6.3 mL). To this mixture at 0° C. was added dropwise a solution of potassium permanganate (0.40 g, 2.52 mmol) in water (9 mL). The mixture solution was stirred at 0 to 15° C. for 2 hours. The reaction mixture was quenched with sodium bisulfite (1.27 g), and hydrochloric acid (2 mol/L, 5 mL) and water (30 mL) were added. The mixture was extracted with 120 mL (40 mL×3) of a mixed solution of trichloromethane and isopropanol (trichloromethane/isopropanol=3/1). The combined organic phase was dried over anhydrous sodium sulfate, and filtered. The filtrate was concentrated under reduced pressure, and 180 mL (30 mL×6) of a mixed solution of toluene and acetonitrile (toluene/acetonitrile=1/1) was added. The solution was concentrated under reduced pressure to give 2-13. ¹H NMR (400 MHz, CD₃OD): δ 5.34 (d, J=2.9 Hz, 3H), 5.06 (dd, J=3.3, 11.2 Hz, 3H), 4.56 (d, J=8.4 Hz, 3H), 4.19-4.06 (m, 9H), 4.04-3.98 (m, 3H), 3.87 (td, J=5.7, 9.9 Hz, 4H), 3.72-3.64 (m, 9H), 3.57-3.50 (m, 3H), 3.39 (br t, J=6.4 Hz, 2H), 3.22 (q, J=6.4 Hz, 12H), 2.51-2.40 (m, 9H), 2.21 (br t, J=7.3 Hz, 6H), 2.14 (s, 9H), 2.03 (s, 9H), 1.94 (d, J=7.9 Hz, 18H), 1.72-1.57 (m, 22H), 1.39 (br s, 12H).

Step K: To a solution of the compound of formula 2-13 (1.00 g, 0.50 mmol) in N,N-dimethylformamide (10 mL) were added N,N-diisopropylethylamine (0.26 g, 1.99 mmol) and O-(7-azabenzotriazol-1-yl)-N,N,N,N-tetramethyluronium hexafluorophosphate (0.23 g, 0.60 mmol). The mixture was stirred and the compound of formula 2-14 (0.23 g, 0.55 mmol) was added. The mixture solution was stirred at 15° C. for 16 h. To the reaction solution were added dichloromethane (50 mL) and water (50 mL), and the layers were separated. The organic phase was washed sequentially with 50 mL of saturated aqueous sodium bicarbonate (50 mL×1), 50 mL of water (50 mL×1) and 50 mL of saturated brine (50 mL×1), dried over anhydrous sodium sulfate, and filtered. The filtrate was concentrated under reduced pressure to give the crude product, which was purified by column chromatography (SiO₂, dichloromethane/methanol (with 0.1% triethylamine)=20/1 to 10/1) to give 2-15. ¹H NMR (400 MHz, DMSO-d₆): δ 7.90-7.82 (m, 6H), 7.78 (br d, J=4.8 Hz, 3H), 7.40-7.26 (m, 10H), 6.91 (br dd, J=3.1, 9.0 Hz, 4H), 5.26 (d, J=3.4 Hz, 3H), 5.03-4.99 (m, 3H), 4.53 (d, J=8.4 Hz, 3H), 4.43 (br d, J=3.8 Hz, 1H), 4.23-4.14 (m, 1H), 4.12-4.02 (m, 9H), 3.92 (td, J=9.0, 11.0 Hz, 3H), 3.78 (s, 6H), 3.77-3.71 (m, 3H), 3.66-3.51 (m, 13H), 3.49-3.41 (m, 4H), 3.11-3.01 (m, 16H), 2.38-2.37 (m, 1H), 2.32 (br s, 9H), 2.14 (s, 9H), 2.08 (br t, J=6.9 Hz, 7H), 2.04 (s, 9H), 1.93 (s, 9H), 1.82 (s, 9H), 1.57-1.46 (m, 22H), 1.31-1.26 (m, 12H).

Step L: To a solution of the compound of formula 2-15 (0.80 g, 0.33 mmol) in dichloromethane (8 mL) were added sequentially triethylamine (67.24 mg, 0.64 mmol), 4-N,N-dimethylaminopyridine (0.12 g, 1.00 mmol) and succinic anhydride (83.13 mg, 0.83 mmol). The mixture solution was stirred at 10° C. for 16 hours. To the reaction solution were added dichloromethane (50 mL), water (30 mL) and saturated brine (30 mL), and the layers were separated. The organic phase was washed sequentially with 30 mL (30 mL×1) of water and 30 mL (30 mL×1) of saturated saline, dried over anhydrous sodium sulfate, and filtered. The filtrate was concentrated under reduced pressure to give the crude product, which was purified by p-HPLC (separation column Waters Xbridge C18 (specifications: 150 mm×50 mm, particle size: 10 μm); mobile phase: [water (10 mM ammonium bicarbonate)-acetonitrile]; elution gradient: 27%-57%, 11 min) to give D01. ¹H NMR (400 MHz, DMSO-d6): δ 7.96-7.69 (m, 9H), 7.33-7.09 (m, 10H), 6.90-6.78 (m, 4H), 5.21 (d, J=3.3 Hz, 3H), 4.97 (dd, J=3.3, 11.2 Hz, 3H), 4.49 (d, J=8.4 Hz, 3H), 4.06-3.97 (m, 9H), 3.91-3.83 (m, 3H), 3.79-3.66 (m, 11H), 3.63-3.45 (m, 18H), 3.02 (br d, J=4.6 Hz, 14H), 2.46-2.37 (m, 4H), 2.35-2.14 (m, 12H), 2.10 (s, 9H), 2.04 (br t, J=7.0 Hz, 6H), 1.99 (s, 9H), 1.88 (s, 9H), 1.77 (s, 9H), 1.57-1.37 (m, 22H), 1.22 (br s, 12H).

D is a residue of the small molecule fragment D01 after chemical reaction, which is bound to a nucleic acid through a covalent bond, and has a structure as shown in the following formula:

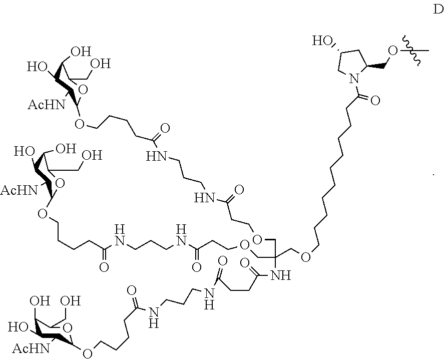

### Example 2: Synthesis of D02

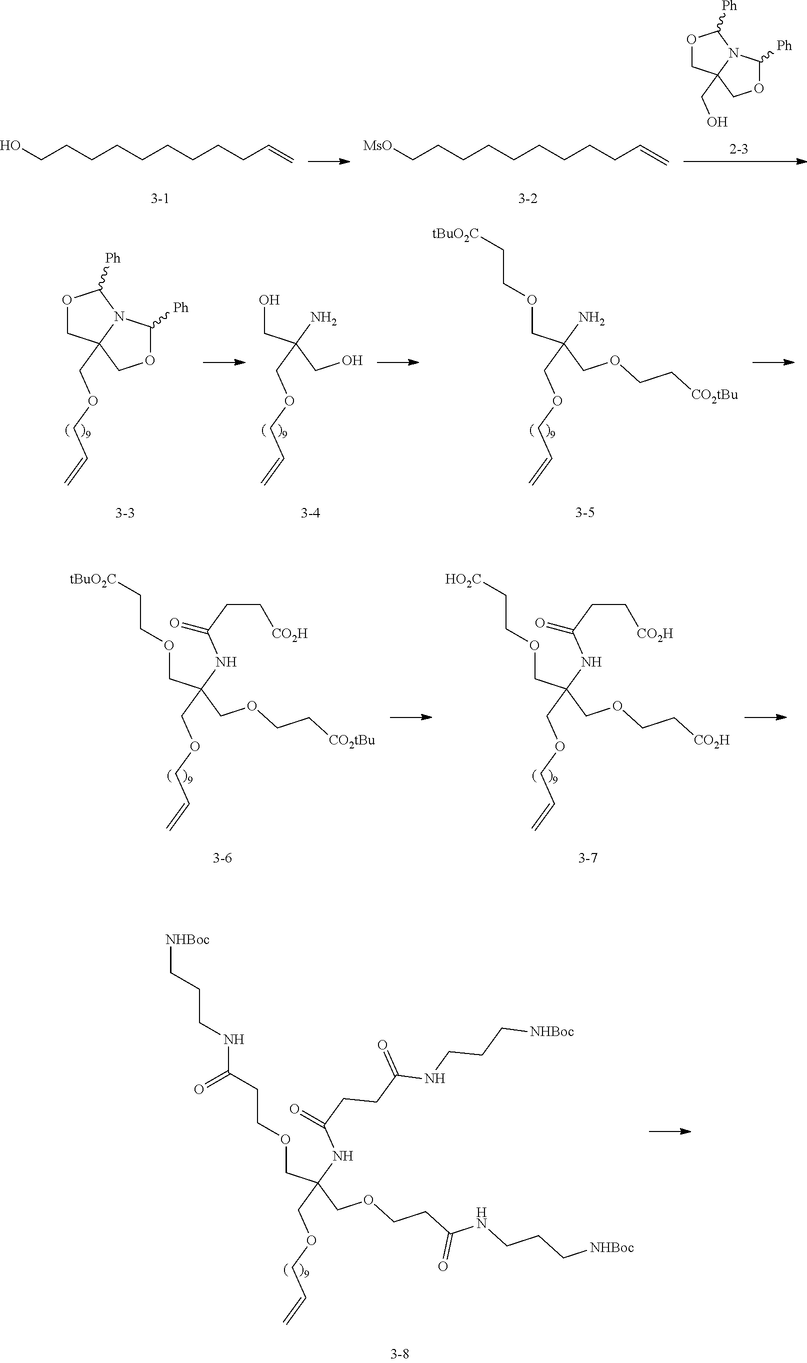

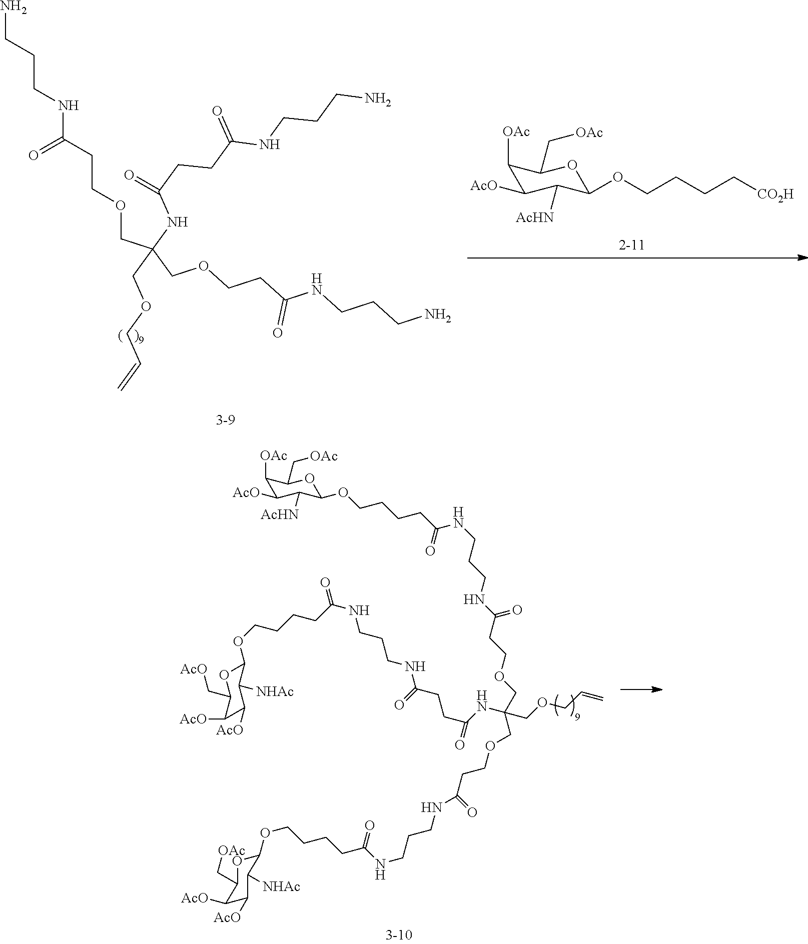

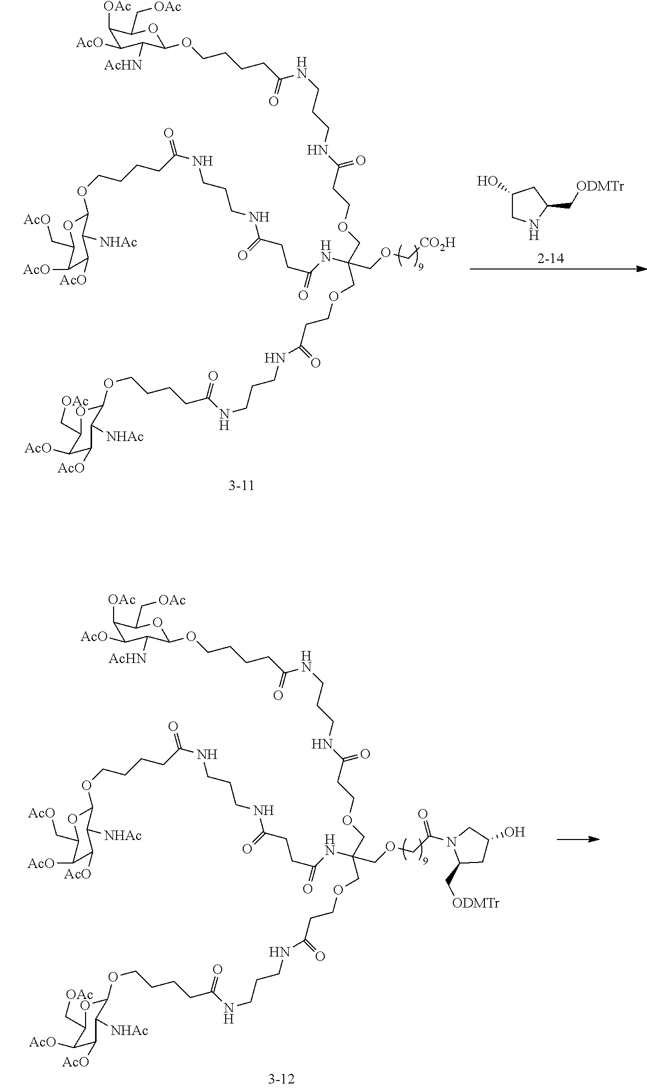

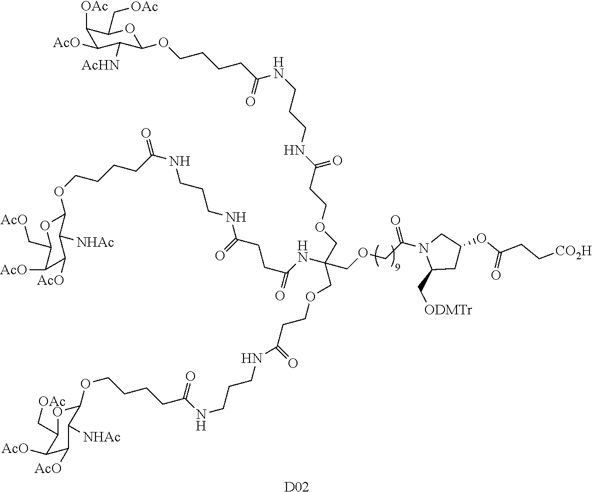

Step A: Compound 3-2 was prepared by referring to compound D01 of Example 1 and replacing 2-1 with 3-1.

Step B: The compound of formula 2-3 (35.70 g, 120.06 mmol) was dissolved in 2-methyltetrahydrofuran (285 mL), and potassium tert-butoxide (17.51 g, 156.07 mmol) was added. The mixture solution was stirred at 85° C. for 2 hours. Then the compound of formula 3-2 (28.40 g, 114.34 mmol) was added. The mixture solution was stirred at 85° C. for 12 h. To the reaction solution was added water (400 mL) and extracted with 800 mL (400 mL×2) of dichloromethane. The combined organic phase was dried over anhydrous sodium sulfate, and filtered. The filtrate was concentrated under reduced pressure to give 3-3.

Step C: The compound of formula 3-3 (35 g, 77.84 mmol) was dissolved in methanol (525 mL), and a solution of hydrogen chloride in methanol (4 mol/L, 175 mL, 750 mmol) was added. The mixture was stirred at 50° C. for 12 hours. The reaction solution was poured into a mixed solution of potassium carbonate (80 g) and methanol (500 mL). The mixture was filtered through a Buchner funnel and the filtrate was concentrated under reduced pressure. The resulting crude product was dissolved in methanol (240 mL), and sodium acetate (12.77 g, 155.68 mmol) and hydroxylamine hydrochloride (5.41 g, 77.84 mmol) were added. The mixture was stirred at 25° C. for 0.5 h. The reaction solution was filtered through a Buchner funnel and the filtrate was concentrated under reduced pressure. The resulting crude product was added to aqueous sodium hydroxide (1 mol/L, 500 mL) and extracted with 500 mL (500 mL×1) of dichloromethane. The combined organic phase was washed sequentially with aqueous sodium hydroxide (1 mol/L, 500 mL) and 500 mL (500 mL×1) of saturated brine, dried over anhydrous sodium sulfate, and filtered. The filtrate was concentrated under reduced pressure to give 3-4. ¹H NMR (400 MHz, CDCl₃): δ 5.81-5.66 (m, 1H), 4.98-4.79 (m, 2H), 3.50-3.37 (m, 4H), 3.36-3.30 (m, 2H), 3.24 (br s, 2H), 2.03-1.91 (m, 2H), 1.53-1.42 (m, 2H), 1.35-1.27 (m, 2H), 1.25-1.13 (m, 10H).

Steps D to I: Prepared by referring to each step of compound D01 of Example 1, respectively.

Step J: The compound of formula 3-10 (20 g, 10.13 mmol) and ruthenium trichloride trihydrate (52.98 mg, 202.61 μmol) were dissolved in a mixed solution of dichloromethane (60 mL), acetonitrile (60 mL) and water (90 mL). Sodium periodate (10.83 g, 50.65 mmol) was slowly added to this mixture solution. The mixture solution was stirred at 25° C. for 3.5 hours. To the reaction solution was added water (500 mL) and the mixture was extracted with 1 L (500 mL×2) of a mixed solution of dichloromethane and isopropanol (dichloromethane/isopropanol=3/1). The combined organic phase was washed with 150 mL (150 mL×1) of saturated aqueous sodium sulfite and concentrated under reduced pressure. The crude product was added to saturated aqueous sodium bicarbonate (500 mL), and the mixture was washed with 1 L (500 mL×2) of dichloromethane. Hydrochloric acid (1 mol/L) was added until the pH was adjusted to 3, and the mixture was extracted with 1.5 L (500 mL×3) of a mixed solution of dichloromethane and isopropanol (dichloromethane/isopropanol=3/1). The combined organic phase was washed with 500 mL of saturated brine (500 mL×1), dried over anhydrous sodium sulfate, and filtered. The filtrate was concentrated under reduced pressure to give 3-11. ¹H NMR (400 MHz, CD₃OD): δ 5.37-5.29 (m, 3H), 5.06 (dd, J=3.4, 11.3 Hz, 3H), 4.61-4.51 (m, 3H), 4.20-3.96 (m, 12H), 3.92-3.82 (m, 3H), 3.70-3.63 (m, 9H), 3.57-3.49 (m, 3H), 3.43-3.36 (m, 2H), 3.28-3.14 (m, 12H), 2.50-2.38 (m, 8H), 2.33-2.24 (m, 3H), 2.24-2.17 (m, 6H), 2.17-2.11 (m, 9H), 2.03-1.99 (m, 9H), 1.98-1.90 (m, 18H), 1.75-1.51 (m, 22H), 1.38-1.27 (m, 10H).

Step K: To a solution of the compound of formula 3-11 (11 g, 5.52 mmol) in dichloromethane (110 mL) were added N,N-diisopropylethylamine (0.71 g, 5.52 mmol), 1-hydroxy-7-azabenzotriazole (1.50 g, 11.04 mmol), O-(7-azabenzotriazol-1-yl)-N,N,N,N-tetramethyluronium hexafluorophosphate (2.52 g, 6.63 mmol) and the compound of formula 2-14 (2.43 g, 5.80 mmol). The mixture was stirred, and then N,N-diisopropylethylamine (2.14 g, 16.56 mmol) was added. The mixture solution was stirred at 25° C. for 12 h. To the reaction solution were added dichloromethane (1 L) and water (500 mL), and the layers were separated. The organic phase was washed sequentially with 500 mL of water (500 mL×1) and 500 mL of saturated brine (500 mL×1), dried over anhydrous sodium sulfate, and filtered. The filtrate was concentrated under reduced pressure to give the crude product, which was purified by column chromatography (SiO₂, dichloromethane/methanol (with 0.5% triethylamine)=30/1 to 10/1) to give 3-12. ¹H NMR (400 MHz, DMSO-d6): δ 7.92-7.73 (m, 9H), 7.38-7.16 (m, 10H), 6.94-6.83 (m, 4H), 5.22 (d, J=3.4 Hz, 3H), 4.97 (dd, J=3.4, 11.3 Hz, 3H), 4.53-4.44 (m, 3H), 4.44-4.35 (m, 1H), 4.21-4.09 (m, 1H), 4.03 (s, 8H), 3.94-3.83 (m, 3H), 3.77-3.68 (m, 9H), 3.58-3.48 (m, 10H), 3.43 (br s, 3H), 3.36-3.28 (m, 4H), 3.03 (br s, 14H), 2.33-2.19 (m, 10H), 2.11 (s, 9H), 2.04 (br t, J=6.9 Hz, 7H), 2.00 (s, 9H), 1.89 (s, 9H), 1.81-1.74 (m, 9H), 1.57-1.38 (m, 22H), 1.37-1.17 (m, 10H).

Step L: D02 was obtained with reference to the preparation of compound D01 of Example 1. ¹H NMR (400 MHz, DMSO-d₆) δ=7.94-7.70 (m, 9H), 7.40-7.08 (m, 10H), 6.95-6.79 (m, 4H), 5.27-5.17 (m, 3H), 4.98 (dd, J=3.3, 11.2 Hz, 3H), 4.50 (d, J=8.4 Hz, 3H), 4.08-3.98 (m, 9H), 3.92-3.85 (m, 3H), 3.78-3.67 (m, 11H), 3.59-3.49 (m, 12H), 3.46-3.36 (m, 6H), 2.95 (br s, 14H), 2.48-2.38 (m, 4H), 2.16 (br s, 12H), 2.11 (s, 9H), 2.05 (br t, J=6.8 Hz, 6H), 2.00 (s, 9H), 1.89 (s, 9H), 1.83-1.72 (m, 9H), 1.57-1.36 (m, 22H), 1.31-1.12 (m, 10H).

### Example 3: Synthesis of D-01-M

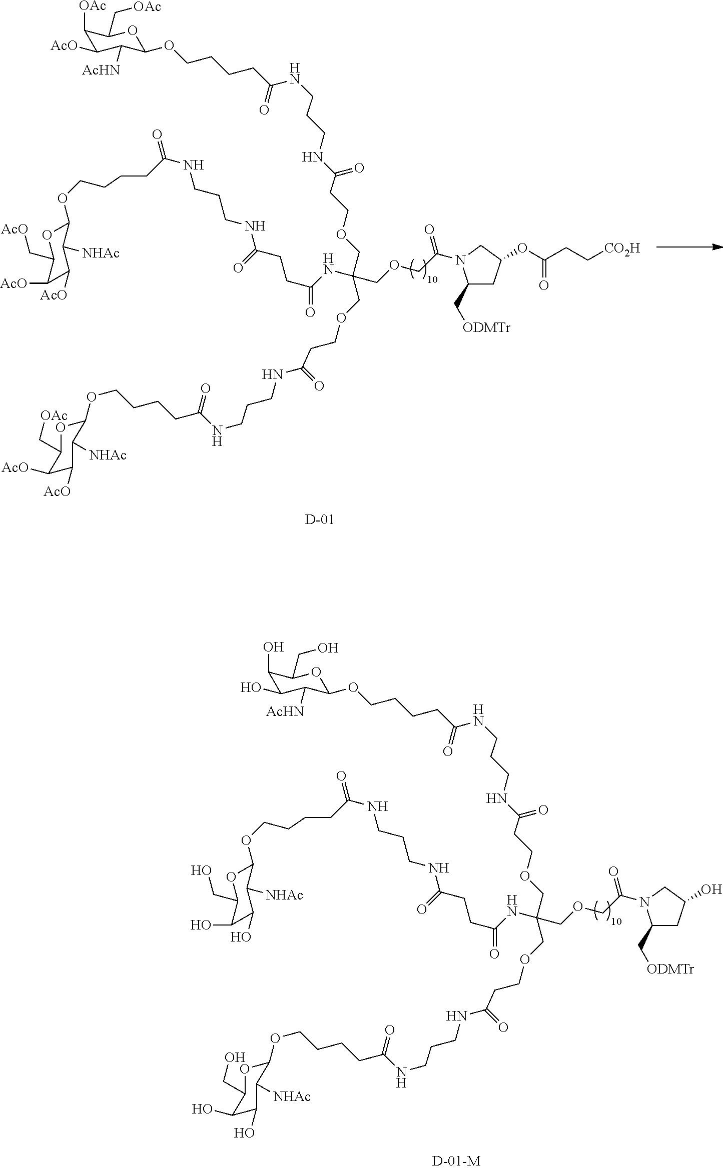

Step A: To a solution of D-01 (100 mg, 39.88 μmol) in acetonitrile (0.7 mL) was added ammonia (30%, 1.4 mL). The mixture solution was stirred at 50° C. for 12 h. The reaction solution was filtered and then purified by p-HPLC (separation column Phenomenex Gemini-NX C18 (specification: 75 mm×30 mm, particle size: 3 μm); mobile phase: [water (0.05% ammonia)-acetonitrile]; elution gradient: 14%-42%, 7 min) to give D-01-M. ¹H NMR (400 MHz, DMSO-d6): δ 7.90-7.57 (m, 9H), 7.38-7.10 (m, 10H), 6.93-6.80 (m, 4H), 4.67-4.52 (m, 6H), 4.52-4.44 (m, 3H), 4.22-4.19 (m, 3H), 3.77-3.62 (m, 15H), 3.58-3.47 (m, 16H), 3.46-3.43 (m, 2H), 3.32-3.25 (m, 6H), 3.12-2.91 (m, 14H), 2.36-2.16 (m, 10H), 2.11-1.99 (m, 7H), 1.85-1.73 (m, 9H), 1.59-1.35 (m, 22H), 1.32-1.13 (m, 12H).

### Assay Example 1. Test of the Binding Affinity of the Compound to Human-Derived Anti-Asialoglycoprotein Receptors

Purpose of the Assay:

The binding affinity of the compound to human-derived anti-asialoglycoprotein receptors (ASGPR) was tested by surface plasmon resonance (SPR) technique, and the kinetic K_{D} value of the compound was used as an indicator to evaluate the binding affinity of the compound to ASGPR, thereby reflecting the ability of the compound to specifically target hepatocytes for delivery of nucleic acid molecules.

Materials of the Assay:

2.1 Protein:

Asialoglycoprotein Receptor Protein, Mouse, Recombinant (His Tag), Sino biological-50083-M07H-50 μg; Asialoglycoprotein Receptor Protein, Human, Recombinant (His Tag), Sino biological-10773-H07H-50 μg

2.2 Reagent:

NiHC 1500M Chip (Xan Tec-SCNihc1500m0720); HEPES (SIGMA-V900477); NaCl (SIGMA-71376); Tween-20 (Aladin-T104863); CaCl2 (SIGMA-C3306-250G); EDTA (SIGMA-3609); NiCl₂ (Energy-chemical-V830089); 10×PBS (Sangon-E607016-0500)

2.3 Materials & Instruments:

Biacore 8 k; Series S CM5 chip (GE Healthcare—BR100530) 96-well plate-250 μL (Greiner-650201); 384-well plate-200 μL (Greiner-781270); 96-well plate-1 mL (Greiner-780201); 96 Microplate foils (GE Healthcare-28975816); 384 Microplate foils (GE Healthcare-BR100577)

3. Steps and Methods of the Assay:

3.1 The two proteins were respectively dissolved in 1×PBS to obtain a solution of 0.25 mg/mL, and the compound to be tested was dissolved in DMSO. The running buffer (10 mM HEPES, 150 mM NaCl, 0.05% Tween 20, 50 mM CaCl₂, 50 μM EDTA) was prepared and filtered through a 0.22 μm membrane.

3.2 The NiHC 1500M Chip was docked into a biacore, and the system was switched to a running buffer.

3.3 Protein labeling: The chip was first treated with 350 mM EDTA at 30 μl/min for 5 min, rinsed with running buffer for 2 min, and then bound with 40 mM NiCl₂ at 30 μl/min for 2 min. A protein diluted to 5 μg/mL with running buffer was then labeled on the chip at a flow rate of 10 μl/min for a final labeling amount of 1500-2000 RU.

3.4 The instrument system was switched to a running buffer containing 2% DMSO.

3.5 The compound with a starting concentration of 2 μM was serially diluted 2-fold with a running buffer containing 2% DMSO to obtain 9 concentrations. The final concentration of DMSO was 2%.

3.6 Testing: For each cycle, the compound was bound for 60 s and dissociated for 180 s at a flow rate of 50 μl/min. The compound was tested in order of concentration from low to high. Before the gradient testing of the compound, 4 cycles of blank (running buffer with 2% DMSO) were introduced, and solvent correction was performed with 1.5%-3.5% DMSO (6 concentrations).

3.7 Analysis of Data:

The data were analyzed with Biacore Insight Evaluation Software, kinetics was analyzed with a 1:1 model, and affinity was analyzed with a steady state affinity model.

| Sample | K_{D} (mol/L) | Kₐ (L/(mol · s)) | K_{d} (1/s) |
|---|---|---|---|
| D-01-M | 1.56 × 10⁻⁸ | 1.08 × 10⁶ | 1.68 × 10⁻² |

Conclusions of the Assay:

In the present assay, the test D-01-M showed good binding affinity in the SPR assay.

The present disclosure demonstrates a delivery platform for oligonucleotide molecules with high efficiency and high hepatocyte targeting: it can bind to a specific, highly expressed ASGPR protein on the surface of hepatocytes, enter the cellular endosome via endocytosis, and work by release into the cytoplasm. The binding affinity constant of this delivery platform to ASGPR is superior to existing technologies. It shows good tissue distribution and metabolic stability in vivo, and is expected to achieve more efficient delivery and efficacy of liver-targeted molecules. The relevant conjugate in the present disclosure shows good activity for HBsAg reduction and long-term efficacy for HBsAg inhibition.

1. A conjugate group, having a structure of formula (I): 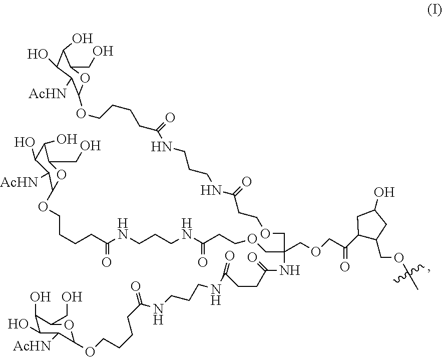
wherein n is selected from an integer of 8 to 12.

2. The conjugate group according to claim 1, having the following structure:

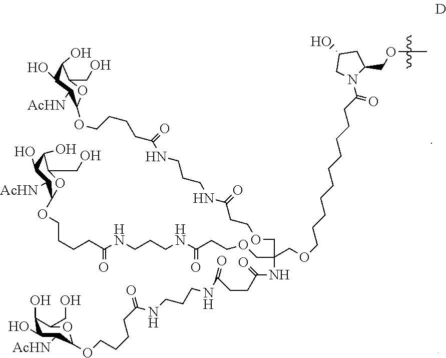

3. A conjugate, comprising the conjugate group according to claim 1, and a therapeutic agent linked to the conjugate group.

4. The conjugate according to claim 3, wherein the therapeutic agent is an expression-inhibitory oligonucleotide.

5. The conjugate according to claim 4, wherein the expression-inhibitory oligonucleotide is an RNAi reagent.

6. The conjugate according to claim 5, wherein the RNAi reagent includes one or more modified nucleotides.

7. The conjugate according to claim 5, wherein the RNAi reagent is a double-stranded siRNA containing a sense strand and an antisense strand.

8. The conjugate according to claim 7, wherein the double-stranded siRNA is linked to the conjugate group at the 5′ end of its sense strand.

9. The conjugate according to claim 4, wherein the expression-inhibitory oligonucleotide is linked to the conjugate group via a phosphate, phosphorothioate, or phosphonate group.

10. A compound, having a structure of formula (II) or formula (III) 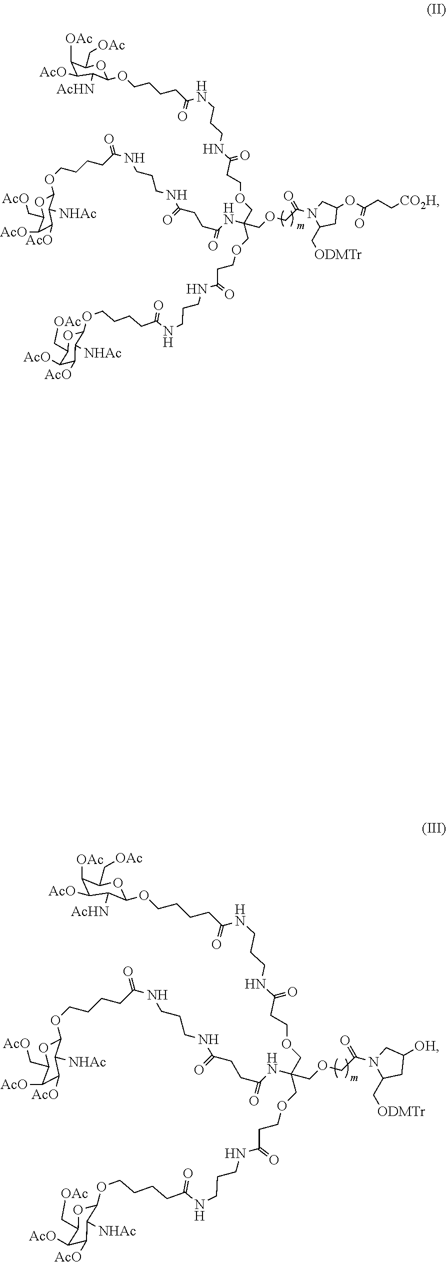
wherein m is independently selected from an integer of 8 to 12, respectively.

11. The compound according to claim 10, having the following structure

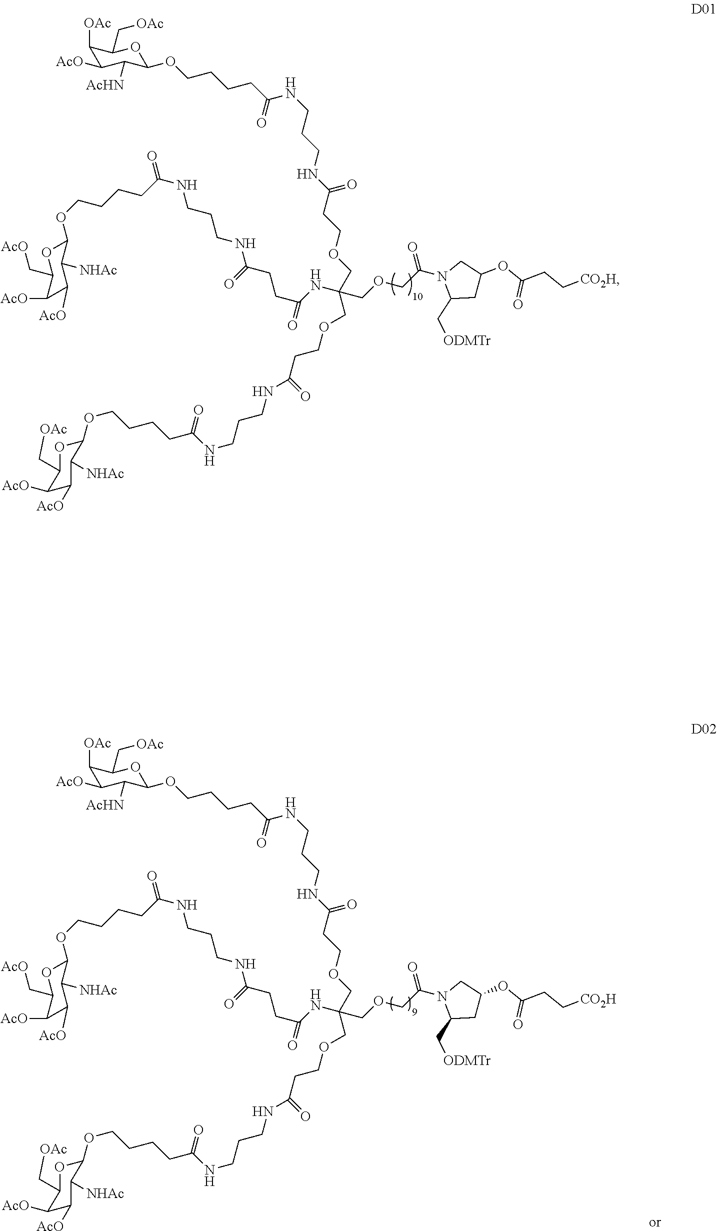

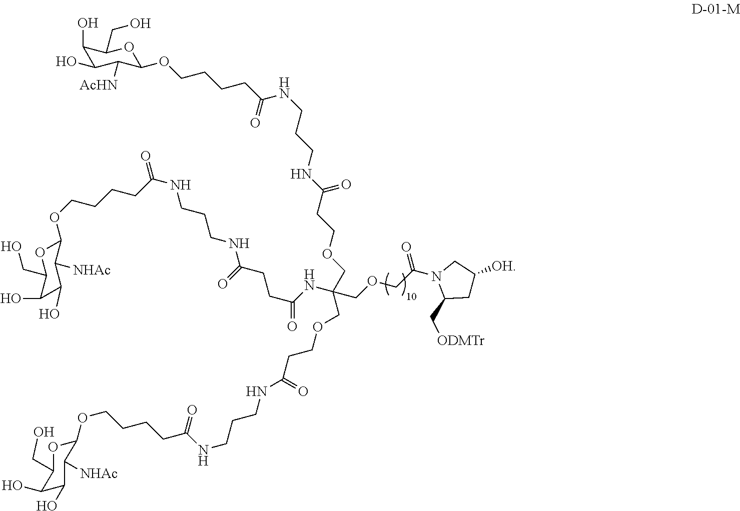

12. A pharmaceutical composition, comprising the conjugate according to claim 3 and a pharmaceutically acceptable carrier or excipient.

13. A method for inhibiting the expression of a target nucleic acid in a subject in need thereof, comprising a step of administering to the subject the conjugate according to claim 3.

14. The method according to claim 13, wherein the target nucleic acid is a nucleic acid from a virus.

15. The method according to claim 14, wherein the target nucleic acid is a nucleic acid from the hepatitis B virus.

16. A method of treating a disease, comprising a step of administering to a subject the conjugate according to claim 3.

17. (canceled)

18. The method according to claim 16, wherein the disease is viral infection.

19. The method according to claim 16, wherein the disease is liver disease.

20. The method according to claim 16, wherein the disease is hepatitis B.
